(19) United States
(12) Patent Application Publication
(10) Pub. No.: US 2009/0018669 A1
(43) Pub. Date: Jan. 15, 2009

(54) Articulating Prosthetic Ankle Joint

(76) Inventors: Ashley D. Agan, Trenton, TX (US); Lena Dixit, Lake Jackson, TX (US); John L. Eakin, Houston, TX (US); Kevin C. Ma, Torrance, CA (US); Richard R. Neptune, Austin, TX (US); Jessica D. Ventura, Austin, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 12/122,006

(22) Filed: May 16, 2008

Related U.S. Application Data
(60) Provisional application No. 60/938,966, filed on May 18, 2007.

(51) Int. Cl. A61F 2/64

(52) U.S. Cl. 623/47; 623/50

(57) ABSTRACT

According to some embodiments, the present description relates to a prosthetic articulating ankle joint. The ankle joint may include a horseshoe shaped component. The horseshoe shaped component may include an anterior gap to allow dorsiflexion, a posterior portion, at least one lateral indentation in the posterior portion to allow eversion or inversion, at least one orifice in a top to allow attachment to a pylon, and at least one orifice in a bottom to allow attachment to a foot. Other embodiments in the description relate to a prosthetic limb including such an ankle joint and a foot and a pylon. In some embodiments, the horseshoe shaped component may be C-shaped. The disclosure also relates to methods for making a prosthetic articulating ankle joint and methods to make a prosthetic limb comprising a prosthetic articulating ankle joint.

### RELATED APPLICATION

This application claims priority to U.S. Provisional Patent Application Ser. No. 60/938,966 filed May 18, 2007, the contents of which are hereby incorporated in their entirety by reference.

### TECHNICAL FIELD

The present disclosure relates to an articulating prosthetic ankle joint. In some embodiments, the joint may be horseshoe-shaped. In other embodiments, it may be capable of use in modifying existing prosthetic devices.

### BACKGROUND

Lower limb amputees worldwide rely on prosthetic devices to avoid confinement to a wheelchair or the use of cumbersome aids such as crutches. However, in order to provide adequate mobility, prosthetic devices preferably need to include some movable or flexible components, such as an artificial ankle joint. These components typically not only have to support the weight of the amputee, but also have to be do so while moving, bending, or flexing. Not surprisingly, ankle joints and similar prosthetic components often fail far more frequently than other prosthetic limb components. Replacing these components can be costly and time consuming, if replacements are even available, particularly for poorer individuals.

For example, Central America has a distinct population of lower limb amputees who do not have access to reliable prosthetic devices. Honduras, one of the poorest countries in Central America, has a distinct population of amputees: young to middle-aged men who have lost limbs as a result of injuries caused by land mines, train accidents, and labor-related activities. This population in particular puts a great amount of stress on their prosthetic limbs during their everyday laboring activities. The prostheses they own simply provide them with the ability to walk, many times without the luxury of comfort or high-tech components. The work environment of most Hondurans can put a high level of stress on a prosthesis, creating the need for repairs and reducing its lifetime. Due to the extent of poverty in Central America, every day's wages are important to the survival of amputees and their families. Taking a day off to visit a prosthetist for repairs is seldom a viable option. For this reason many amputees choose to ‘fix’ their limbs themselves with wires and string, thus, allowing them to continue working.

In addition to laboring conditions, the environment can also age the prosthesis. Central America has very rugged terrain and a humid climate. The combination of moisture and dust contributes to the rapid degradation of cosmetic prosthetic covers, as well as to the corroding of joints, like prosthetic knee joints. Since 1999, A non-profit organization called Central American Medical Outreach (CAMO), founded by Kathy Tschiegg, has taken the initiative to alleviate the lack of medical services available to Central Americans. This group collects donated prosthetic components, such as prosthetic limbs, orthotics and braces, from companies and individuals in the United States and distributes them to their orthotics and prosthetics laboratory in Santa Rosa de Copan, Honduras. The purpose of the non-profit organization has been to provide quality medical services, education, and medical supplies to Central Americans. To support 15 various medical programs in Honduras, CAMO receives donated medical supplies, funding, and volunteer work. The organization has been tremendously successful in gathering the basic components of prostheses: feet (mostly solid ankle cushion heel (SACH)), pylons, and socket materials. The major components of prosthetic legs are readily available at this facility, and a resident prosthetist fabricates, fits, and aligns complete prostheses for the amputees. This effort provides many amputees with a minimal solution—a prosthetic limb that gives them basic functionality.

However, many of the donated prosthetic parts have been previously used and are somewhat primitive compared to modern prosthetics. In addition, many of the prosthetic limbs initially belonged to diabetic Americans and therefore were intended for individuals leading considerably different lifestyles than Honduran laborers. Due to the strenuous activities the limbs are subjected to, amputees in Honduras have prosthetics that are more prone to breakage or discomfort. In 2006, CAMO provided 14 prosthetic legs to Central American amputees. The more striking figure is the 87 changes and repairs made. Though some of these reparations are normal and expected refittings, others are a result of harsh Honduran living conditions. This provides an incentive to create a design that will be resistant to labor and environmental associated wear. Further, due to the rugged, mountainous terrain of Central America, amputees could benefit from articulating ankles. This would allow for a more natural gait through the support of plantar and dorsiflexion, as well as inversion and eversion.

Additionally, there is a need for low-cost prosthetic devices. For example, Central America consists of a string of seven low income, developing countries located immediately south of Mexico. These countries include Belize, Costa Rica, El Salvador, Guatemala, Honduras, Nicaragua, and Panama. The Gross Domestic Product (GDP) per capita for these countries ranges from $3,000 to $12,000. The United States has a GDP per capita of $43,500. Comparison of values of GDP per capita illustrates the vast difference in the standards of living and quality of life of Central Americans compared to citizens of the United States. It is also informative to note that over half of the population of Honduras lives below the poverty line, a value that is defined separately by each country.

The problem Honduras faces today is present in many other low-income countries as well. According to a survey done by Prosthetics and Orthotics International, eighty percent of the world's disabled population lives in “abject poverty in remote rural areas.” A new, successful design for a cheap, manufacturable prosthetic limb has the potential to help with conditions in other countries as well and to provide cost-effective, simple or durable prosthetic options even in countries, such as the US, where more complex devices are more readily available.

A prosthetic ankle may also allow amputees in the US or other countries with more complex devices to benefit from a cheaper prosthetic device that might, for example, allow the amputee to engage in vigorous activity that might break the prosthetic ankle.

Endolite manufactures a multiflex ankle that can be added to existing feet (e.g., prosthetic feet). It functions on the basis of a rubber snubber that provides resistance to ankle flexion in 360°. Though the ankle does provide some flexion, the snubber is susceptible to cracks and breaks. In addition, patients often felt unstable on the Endolite ankle. It caused their gait to be wobbly due to excessive flexion in all directions.

### SUMMARY

The present disclosure overcomes limitations in the art and provides prosthetic articulating ankle joints that comprise features such as: durability, suitability for use in rugged outdoor environments, heat and humidity resistance, ease of manufacture and are low cost.

The disclosure relates to a prosthetic articulating ankle joint comprising a horseshoe shaped component that comprises a means to provide dorsiflexion, a means to provide eversion and/or inversion and/or torsion, a means for attachment to a pylon, and a means for attachment to a prosthetic foot. In some embodiments, the horseshoe shaped component may be C-shaped.

According to one embodiment, the disclosure relates to a prosthetic articulating ankle joint. The ankle joint may include a horseshoe shaped component. The horseshoe shaped component may include an anterior gap to allow dorsiflexion, a posterior portion, at least one lateral indentation in the posterior portion to allow eversion and/or inversion and/or torsion, at least one orifice in a top to allow attachment to a pylon, and at least one orifice in a bottom to allow attachment to a foot. Some embodiments of the disclosure relate to a prosthetic limb and comprise a prosthetic articulating ankle joint and a prosthetic foot and a pylon.

In some embodiments, the disclosure provides a prosthetic articulating ankle joint where the horseshoe shaped component may be a C-shaped component. In some embodiments, the C-shaped component may comprise a top anterior projection, a bottom anterior projection, and a gap between the top anterior projection and the bottom anterior projection. In some embodiments, the top and bottom anterior projections of the C-shaped component prevent excessive dorsiflexation. In some embodiments, the top and bottom anterior projections of the C-shaped component reduce excessive dorsiflexation. In some embodiments, the gap between the top- and bottom-anterior projections closes upon itself upon exposure of the prosthetic articulating ankle joint large forces.

In some embodiments, a prosthetic articulating ankle joint of the disclosure comprises an open interior wherein the anterior gap allows compression during dorsiflexation.

In some embodiments, orifices, located in the bottom to fit a prosthetic foot and located in the top to fit a pylon, may have a universal-fit design. Thus, any prosthetic foot or any pylon may be used in conjunction with a prosthetic articulating ankle joint of the disclosure. In some embodiments, a prosthetic foot that may be a non-articulating foot may be used, for example, a solid ankle cushion heel foot (SACH foot). In other embodiments, a prosthetic foot may be an articulating foot may be used. Some exemplary articulating prosthetic feet include TruStep, Venture, Tribut and/or TruPer. In yet other embodiments, an energy storing prosthetic foot such as a Flex-foot may be used. One of skill in the art will recognize that a prosthetic articulating ankle joint of the disclosure may be used in conjunction with any kind of prosthetic foot and that one is not limited by the exemplary feet described herein. One of skill in the art will also recognize that a prosthetic articulating ankle joint of the disclosure may be used in conjunction with any kind of pylon as well.

In some embodiments a prosthetic articulating ankle joint of the disclosure may be comprised of one or more materials such as a high density polyethylene (HDPE), acrylonitrile butadiene styrene (ABS), Nylon, white Delrin, polypropylene, and/or polyethylene.

The present disclosure also relates to a prosthetic limb that may comprise a prosthetic foot, a pylon, and a horseshoe shaped prosthetic articulating ankle joint as described herein. In some embodiments, a prosthetic limb of the disclosure comprises a horseshoe shaped prosthetic articulating ankle joint that may be C-shaped.

In some embodiments, the present disclosure provides an articulating ankle for a prosthetic limb. Some embodiments provide a durable prosthetic limb or ankle joint. Some embodiments provide an inexpensive prosthetic limb and/or a prosthetic ankle. In some embodiments, the present disclosure provides a prosthetic limb or an ankle joint able to endure the wear and tear associated with labor-intensive employment. Some embodiments provide a prosthetic limb or an ankle suitable for mountainous conditions. In some embodiments, the disclosure provides a weather-resistant prosthetic limb or an ankle. Some embodiments provide a prosthetic limb or an ankle suitable for use in rugged outdoor conditions.

Some embodiments relate to a prosthetic limb or an ankle joint that may be easily manufacturable. Some embodiments provide a prosthetic limb or an ankle that is resistant to fracture or deformation due to fatigue and/or overuse. Some embodiments relate to a prosthetic limb or an ankle that requires minimal repair.

In some embodiments the disclosure relates to an ankle that returns to its initial state after movement or flexion. The disclosure relates to a prosthetic limb or an ankle that can hold and sustain heavy loads, for example, a load of up to 250 lbs may be held. Thus in some embodiments, about 10 lbs, about 20 lbs, about 30 lbs, about 40 lbs, about 50 lbs, about 60 lbs, about 70 lbs, about 80 lbs, about 90 lbs, about 100 lbs, about 110 lbs, about 120 lbs, about 130 lbs, about 140 lbs, about 150 lbs, about 160 lbs, about 170 lbs, about 180 lbs, about 190 lbs, about 200 lbs, about 210 lbs, about 220 lbs, about 230 lbs, about 240 lbs, and about 250 lbs. may be held.

The disclosure also provides an ankle joint that can universally fit between a pylon and/or a prosthetic foot. For example, in some embodiments the ankle joint is designed to fit any pylon and/or any prosthetic foot that is available.

In some embodiments the present disclosure provides an articulating prosthetic ankle joint with a minimum lifetime of approximately 20 years. Some embodiments provide an articulating prosthetic ankle joint with a minimum lifetime of approximately 5 years, of approximately 10 years, of approximately 15 years, of approximately 20 years.

### BRIEF DESCRIPTION OF THE DRAWINGS

The present disclosure may be better understood through reference to the following detailed description taken in conjunction with the following Figures.

FIG. 1 shows the normal human ankle in its neutral orientation, dorsiflexion, and plantar flexion.

FIG. 2 shows the normal human ankle at its neutral position, eversion and inversion.

FIG. 3 shows a sagittal cross-sectional view of a SACH foot, a type of non-articulating foot.

FIG. 4 shows various multi-axial feet.

FIG. 5 shows an energy-storing foot.

FIG. 6 shows example prosthetic ankles with posterior indentation, according to an embodiment of the present invention.

FIG. 7 shows an example prosthetic ankle in a C-shaped horseshoe configuration, according to an embodiment of the present invention.

FIG. 8 shows a SolidWorks extrusion geometry, according to an embodiment of the present invention.

FIG. 9 shows the angle of articulation, according to an embodiment of the present invention.

FIG. 10 shows the fully fabricated C-shape ankle before assembly in an isometric view, according to an embodiment of the present invention.

FIG. 11 shows the rear portion of the ankle in an isometric view, according to an embodiment of the present invention.

FIG. 12 shows an isometric front view of the ankle, according to an embodiment of the present invention.

FIG. 13 illustrates the variable inner and outer diameters of the ankle in an isometric view, according to an embodiment of the present invention.

FIG. 14 shows an isometric view of the assembly of the superior portion of the ankle, according to an embodiment of the present invention.

FIG. 15 shows an isometric view of the assembly of the inferior portion of the ankle, according to an embodiment of the present invention.

FIG. 16 illustrates an isometric view of how the ankle is fixed to a prosthetic pylon, according to an embodiment of the present invention.

FIG. 17 illustrates an isometric view of how the ankle is fixed to a prosthetic foot according to an embodiment of the present invention.

### DETAILED DESCRIPTION

The current disclosure, in one embodiment, relates to an articulating prosthetic ankle joint. The ankle joint may be horseshoe shaped. The ankle joint may be in a C-shaped horseshoe configuration. It may further be part of a prosthetic device, such as a prosthetic limb.

A prosthetic foot/prosthetic ankle composition greatly affects range of motion for both walking and running and are very important for amputee comfort. Like the natural foot/ankle complex, the prosthetic counterparts must be able to adapt to different walking and running speeds, habits, and various terrains. The size of a prosthetic foot and/or a prosthetic ankle, are generally designed to be the same size as the original foot.

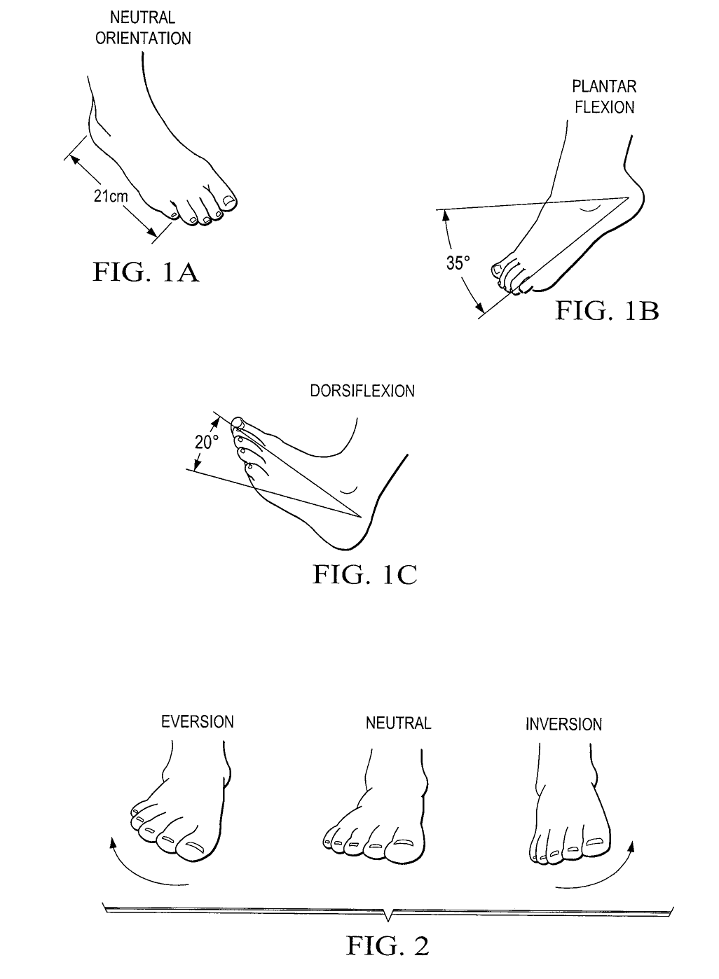

Prosthetic feet and ankle complexes attempt to mimic natural ankle flexion. The normal human ankle range of motion (ROM) includes plantar flexion, dorsiflexion, inversion, and eversion. Plantar flexion is the motion of the toe moving away from the body and dorsiflexion is the toe moving toward the body (FIG. 1). Inversion is the bottom of the foot rolling toward middle of the body, while eversion is the bottom of the foot facing outward (FIG. 2).

Different prosthetic ankle joints may have different stiffness. Selection of appropriate stiffness may be based on individual uses and activities, body weights, and usage. Further, the geometry and material selected for a prosthetic ankle may affect the lifetime of the component.

One embodiment of the disclosure, shown in FIG. 6, provides a horseshoe-shaped articulating prosthetic ankle joint. The joint may contain an open interior to allow compression during movement, particularly dorsiflexion. The joint may also include indentations on the posterior side or sides to allow inversion and eversion. Further, the joint may include bolt holes or other similar features on the top or bottom to allow connection to a foot and/or a pylon. In some embodiments, the bolts and bolt holes may have a universal-fit design and may be connected to any foot or any pylon that may be available. In some embodiments, a universal-fit design comprises bolts and bolt holes that are designed to fit into any pylon or any prosthetic foot.

According to another embodiment, shown in FIG. 7, the ankle joint may be a C-shaped horseshoe. The top and bottom anterior projections in this embodiment help prevent excess dorsiflexion by the ankle.

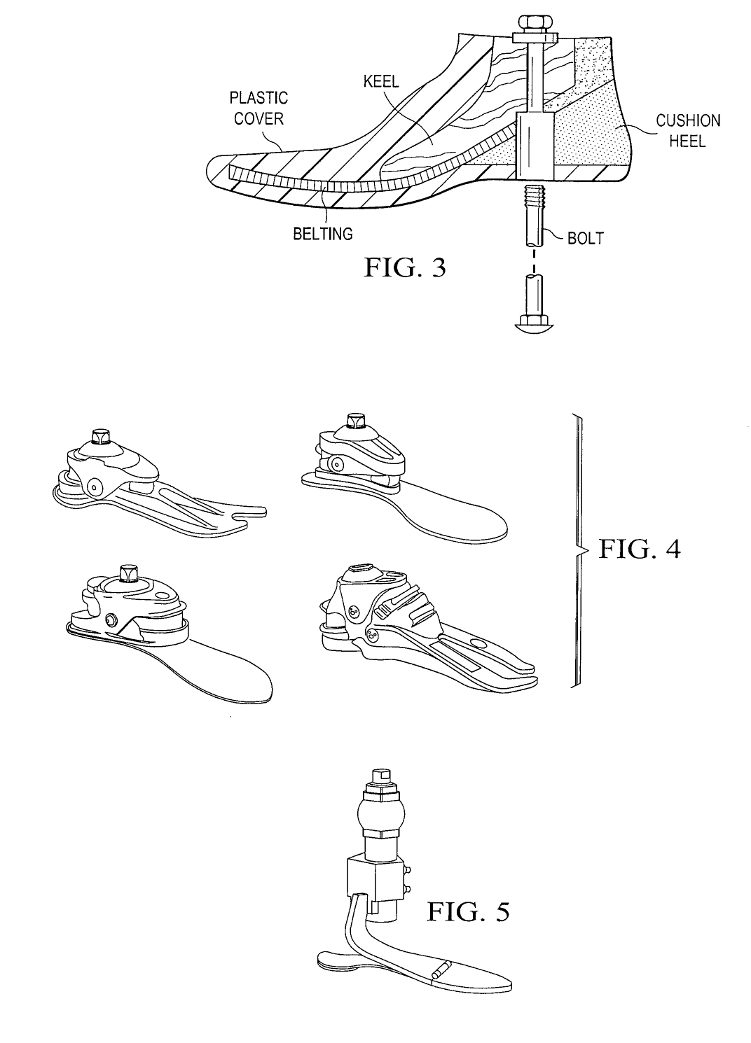

In one embodiment, a non-articulating foot may be used. For example, SACH (solid ankle cushion heel) feet may be used. These are considered non-articulating feet because they lack an ankle joint. The SACH foot's compressible heel works to provide a pseudo-plantar flexion during walking. In addition, the foot contains a solid wooden keel which does not allow for much lateral movement, yet it is quite stable (FIG. 3). In some embodiments, a non-articulating foot may be used with an articulating ankle joint of the present disclosure to provide a prosthetic limb.

In some embodiments, an articulating foot may be used. The articulating foot may include an ankle joint and so increases shock absorption at heel strike, promotes stability at the knee, and allows the foot to conform to uneven surfaces. Further, there are several multi-axial feet on the market that support articulation. Examples include the TruStep, Venture, Tribute, and TruPer, which are all made by College Park Industries (FIG. 4). These multi-axial feet employ highly complex designs to achieve advantages such as torque and shock absorption, as well as ability to conform to uneven terrain. Because of the complexity of the design, these feet cost considerably more than SACH feet. Due to a greater amount of components, they may be heavier than SACH feet, which may be a disadvantage in some embodiments. In some embodiments, an articulating foot may be used with an articulating ankle joint of the present disclosure to provide a prosthetic limb.

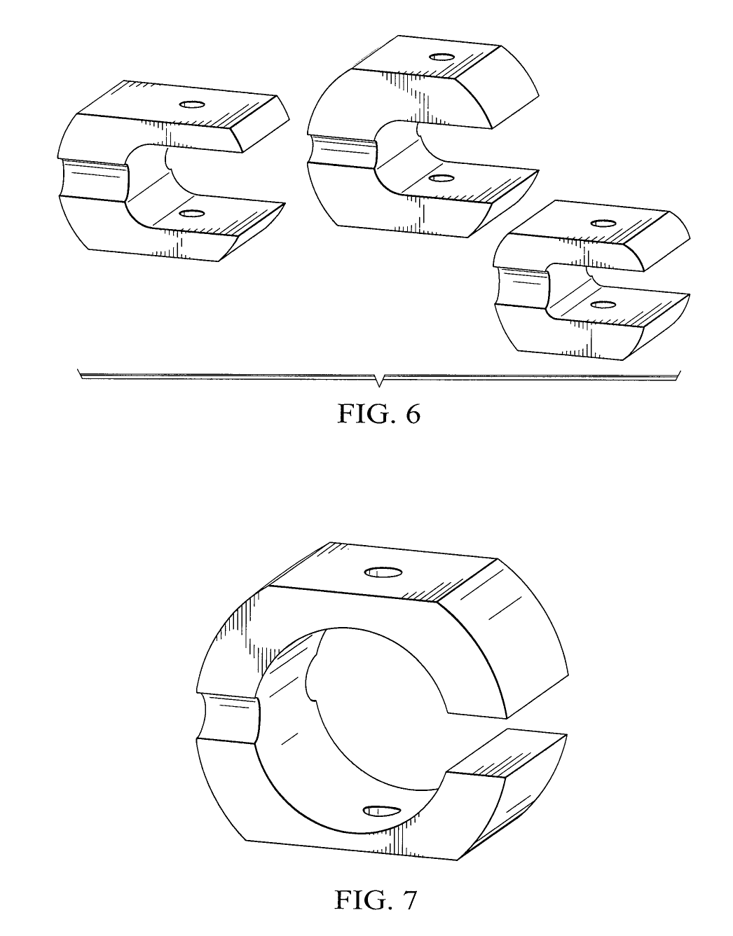

Energy storing feet, like the Flex-Foot by Ossur (FIG. 5), also provide foot flexion. Its design is unlike conventional feet and includes two main carbon fibers leaves. The second leaf on the bottom of the foot serves to attenuate the shock of heal-strike and force the foot forward. The larger, main leaf serves to provide dorsiflexion (FIG. 1) during stance phase, and then exerts force through extension during push-off. Though this design lacks cosmetics, it is very lightweight. However, this type of energy storing feet may cost approximately ten times more than a SACH foot. In some embodiments, an energy-storing foot may be used with an articulating ankle joint of the present disclosure to provide a prosthetic limb.

In some embodiments, an articulating ankle of the present disclosure may be movable in more than one direction. In some other embodiments, such as those designed to allow walking uphill, an articulating ankle of the present disclosure may be movable to simulate dorsiflexion.

In some embodiments, an articulating ankle of the present disclosure may return to the resting state while no force is applied on the ankle. This allows the ankle to be flexible and have spring action to accommodate different angles of inclines and terrains without changing its shape permanently. This characteristic may also give an articulating ankle of the present disclosure its energy return property.

According to another embodiment, an articulating ankle of the present disclosure may be designed to allow the amputee to perform hard labor, such as involving pushing a heavy load uphill. The ankle may be robust and sturdy in order to provide the amputees adequate support to perform hard labor. In some embodiments, an articulating ankle of the present disclosure may be able to endure an estimated minimum load of 250 lbs. to enable amputees to perform hard labor. In some embodiments, an articulating ankle and/or a prosthetic limb of the present disclosure may be able to endure a minimum load of about 250 lbs-about 1000 lbs, for example about 250 lbs, about 275 lbs, about 300 lbs, about 325 lbs, about 350 lbs, about 400 lbs, about 424 lbs, about 450 lbs, about 500 lbs, about 550 lbs, about 600 lbs, about 650 lbs, about 700 lbs, about 750 lbs, about 800 lbs, about 850 lbs, about 900 lbs, about 950 lbs to about 1000 lbs may be the minimum weight loads endured. In some embodiments, loads of about 10 lbs-about 100 lbs, about 100 lbs-about 250 lbs, about 250 lbs-about 300 lbs, about 300 lbs-about 500 lbs and about 500 lbs-about 1000 lbs may be supported.

According to some embodiments, a prosthetic ankle of the disclosure may be designed to be an add-on feature between an existing SACH foot and pylon. As an add-on, a prosthetic ankle of the disclosure may fit universally onto all foot designs or all foot designs of a particular type, such as SACH foot designs. For example, an ankle may accommodate SACH foot bolts (M8×1.25×50) for attachment to the foot and pyramid. A universal-fit ankle design may also save money and resources in manufacturing and/or fabricating a prosthetic ankle and/or a prosthetic limb of the disclosure.

In some embodiments, an articulating ankle may be designed for constant exposure to the outdoors and heavy, cyclic usage through its lifespan. Therefore, the material used to make an ankle design may be able to endure common weather extremes, such as direct sunlight and high humidity. The ankle may also resist material fatigue to retain its endurance and flexibility.

In some embodiments, the material used may be selected based on one or more of the following properties: tensile strength, impact strength, coefficient of thermal expansion, weather resistance, machinability, cost, and modulus of elasticity. Some non-limiting exemplary materials that may be used include: high density polyethylene (HDPE), acrylonitrile butadiene styrene (ABS), Nylon, White Delrin, Polypropylene and the like. In some embodiments, an articulating ankle of the disclosure may be made from polyethylene. Other plastics may be used as well. One or more of the forgoing materials may be used in the manufacture and fabrication of an ankle joint or a prosthetic limb of the disclosure. Relevant properties of some of the materials described herein are provided in Table 1.

TABLE 1
Material Properties

| | Polyethylene | ABS | Nylon | White Delrin | Polypropylene |
|---|---|---|---|---|---|
| Tensile | 6500 | 5500 | 12000 | 10000 | 5000 |
| Strength (psi) | | | | | |
| Impact Strength | No break | 7.5 | 0.4 | 2.3 | ~1 |
| (ft-lbs/in) | | | | | |
| Hardness | D67 | R105 | R115 | M94 | ~R80 |
| Coeff. Thermal | ~10⁻⁴ | ~10⁻⁵ | ~10⁻⁵ | ~10⁻⁵ | ~10⁻⁴ |
| (in/in/F.) | | | | | |
| Weather | Unstable in UV | Good | Bad | Unstable in UV | Unstable in UV |
| Resistance | | | | | |
| Machinability | Standard | Easy | Easy | Easy | Limitation |
| Cost per foot | $13.00 | $12.65 | $8.83 | $36.00 | $5.92 |
| D = 1.5″ | | | | | |

Important specifications for each material were obtained from material manufacturers and compared with the assistance of a Pugh Chart as shown in Table 2.

TABLE 2
Pugh Chart

| | | Concepts | | | | |
|---|---|---|---|---|---|---|
| | | Datum | | | White | |
| | | (Polyethylene) | ABS | Nylon | Delrin | Polypropylene |
| Criteria | Tensile | 0 | −1 | 1 | 1 | −1 |
| | Strength | | | | | |
| | Impact | 0 | −1 | −2 | −2 | −2 |
| | Strength | | | | | |
| | Thermal | 0 | 1 | 1 | 1 | −1 |
| | Expansion | | | | | |
| | Weather | 0 | 1 | −1 | 0 | 0 |
| | Resistance | | | | | |
| | Machinability | 0 | 1 | 1 | 1 | −1 |
| | Flexibility | 0 | −1 | −1 | −1 | −1 |
| | Cost | 0 | −1 | −1 | −2 | 0 |
| | Sum of + | | 3 | 3 | 3 | 0 |
| | Sum of − | | −4 | −5 | −5 | −6 |
| | TOTAL | 0 | −1 | −2 | −2 | −6 |

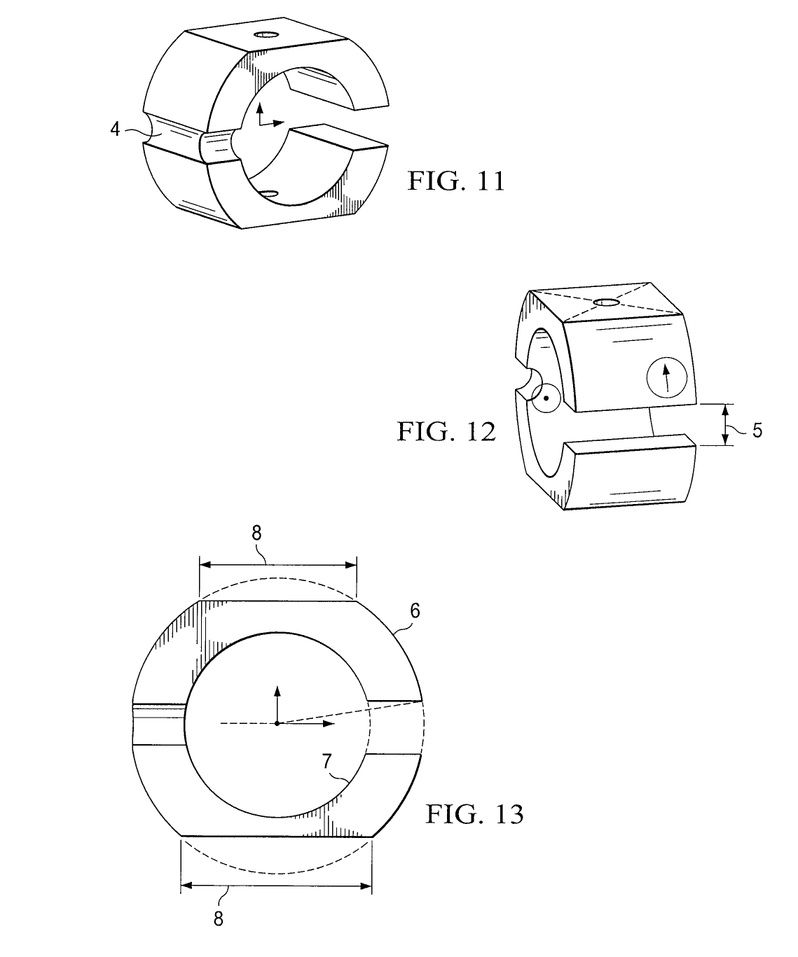

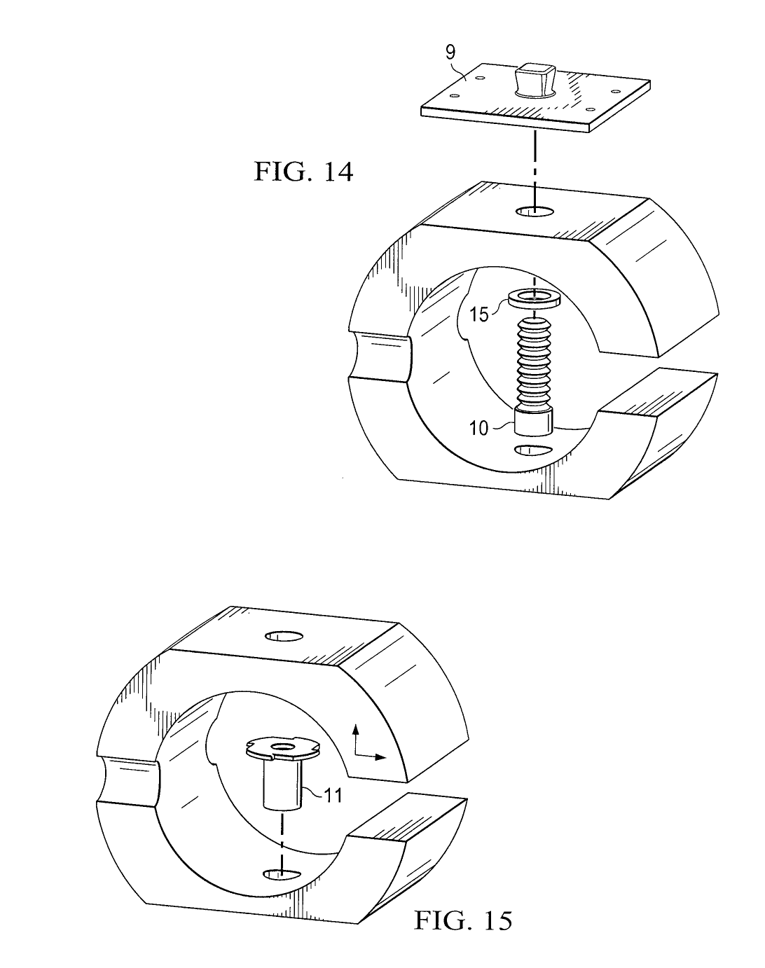

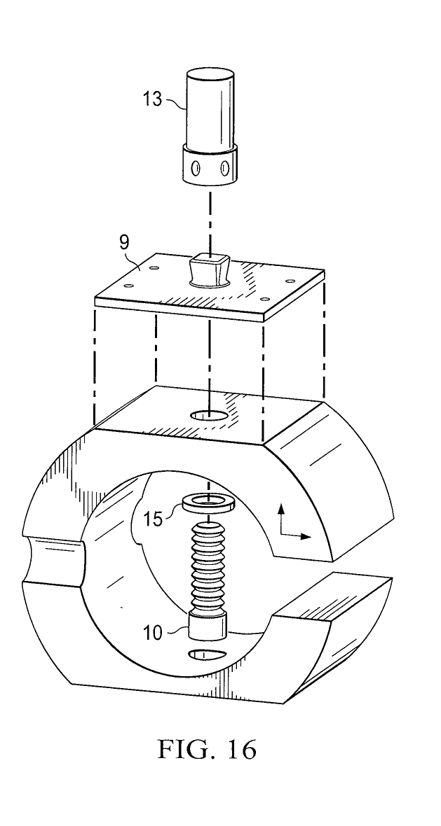

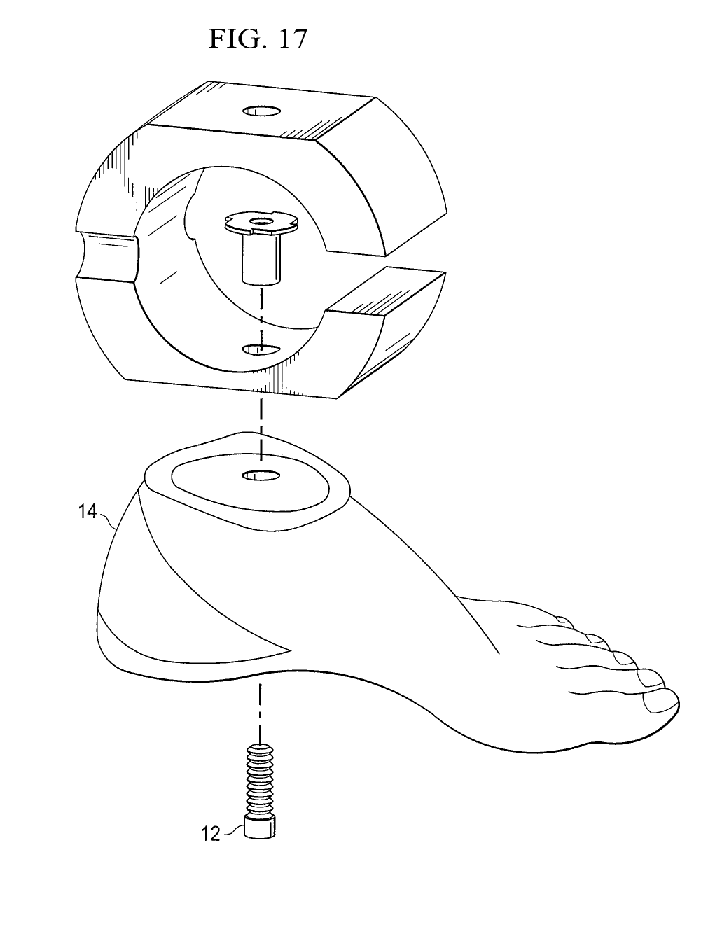

Some embodiments of the novel articulating ankle joint are described below with reference to the indicated drawings. FIG. 10 shows the basic geometry of the C-shaped articulating ankle joint. The device may be constructed from a plastic such as Delrin, however, other materials may also be used to achieve functionality. The isometric view of an ankle shown in FIG. 10 illustrates the superior bolt hole 1, and the inferior bolt hole 2, which may be used to bolt an ankle to a prosthetic pylon and a prosthetic foot, respectively. The lateral notches, indentations or “bites” 3 serve to promote inversion, eversion, and torsion of the ankle. In FIG. 11, the posterior side of an ankle 4 may be sanded down to ease fabrication of the lateral bites. FIG. 12 portrays the gap 5 of an ankle which promotes articulation and closes upon itself when the ankle is exposed to large forces. FIG. 13 demonstrates how an ankle dimensions can be changed to fit the specific needs of the amputee. The outer diameter 6 generally ranges from about 1.5″ to about 3.5″. This value may equal the diameter of the plastic rod used to fabricate an ankle. The outer dimensions correlate with the inner diameter 7 and vice versa because unique ankle articulation and functionality is achieved using different permutations of these values. The inner diameter values generally range from about 1.25″ to about 2.5″. These values may be dependent on the drill bit sizes that are available to the prosthetist fabricating the ankle. FIG. 13 also shows that the top and bottom parts of a C-shaped ankle 8 may be leveled off to ensure fixation to a pylon and/or a foot, respectively. These surfaces may or may not be parallel, depending on how and by whom the device was fabricated. Typically, these surfaces may be flattened using a sanding machine or a milling machine. FIG. 14 illustrates the washer/bolt/pyramid system that may be assembled on the top portion of an ankle. A curved washer 15 may be placed underneath the bolt hole and a bolt 10 with a thread length of 1.5″ is placed through the washer and bolt hole. On top of the ankle, a prosthetic pyramid 9 may be screwed tightly to the bolt using an Allen wrench. FIG. 16 shows how the pylon 13 is attached to the pyramid on top of the ankle. The bolt/tee nut system that may be assembled on the bottom portion of the ankle is shown in FIG. 15. A tee nut 11 may be placed with teeth (not visible in picture) pointing downward into the inferior bolt hole of the ankle. To complete assembly of the lower part of an ankle, a bolt 12 with thread length of 1.5″ may be placed through a prosthetic foot 14 shown in FIG. 17 and tightened with an Allen wrench.

### EXAMPLES

Aspects of the invention may be better understood through reference to the following examples. These examples, in whole or in part, are not intended to illustrate to entire scope of the invention. Appropriate variations and modifications may be made.

### Example 1

### Prosthetic Ankle Design

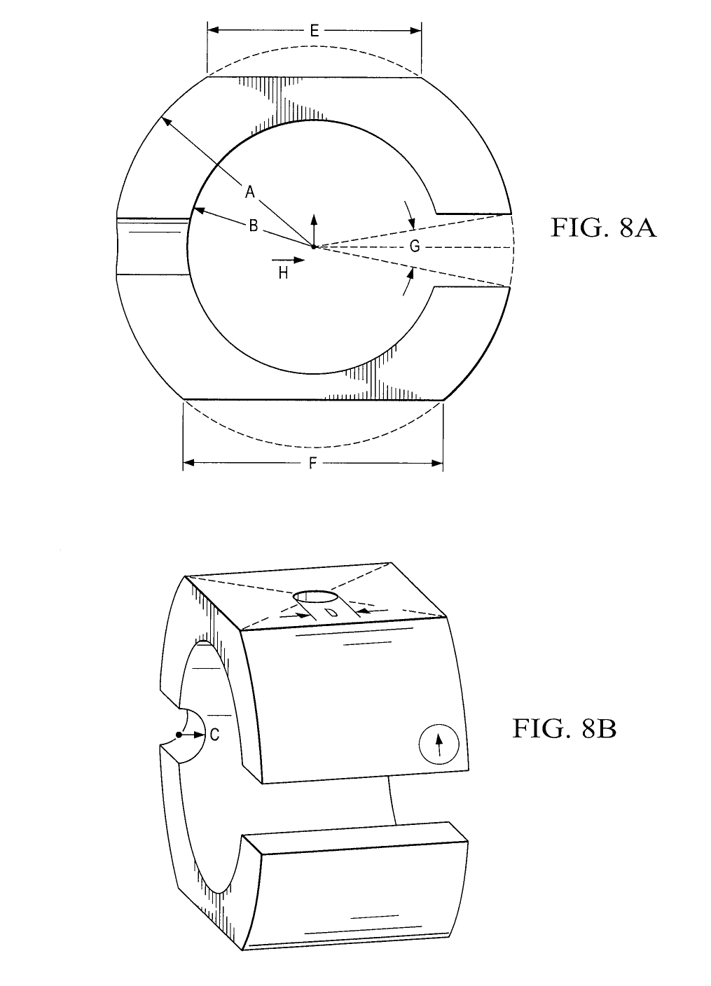

The first step toward producing a functional prototype was creating a virtual model of the “horseshoe” design. This basic visualization provided a foundation to optimize the performance of the device via changes in its geometry and material makeup. To make these changes easier, variables were defined for every dimension-of-interest in the device. By changing the dimensions of the device, many models were rapidly and easily created. The pictures in FIG. 8 illustrate the shapes used to extrude this geometry.

In FIG. 8, outer diameter A is the diameter of the circular, flat faces of the device. These make the left and right sides of the ankle. Inner diameter B is the diameter of the whole cut through the center of the ankle. Indentation radius C (also called bite radius C) is the radius of the notches cut out of the lateral portions of the posterior section of the device. Screw diameter D is the diameter of the wholes cut through the center of the top and bottom faces. Top length E is the anteroposterior length of the top face of the device. Bottom length F is the anteroposterior length of the top face of the device. Angle G is the angle measured from the center of the device, which is used to cut the gap from the anterior section. This allows some articulation to occur. Distance from quarter back H is the distance of the inside whole from one quarter inch back from the center axis of the device. This variable was used only in the second set of tests. The width of the device was not defined as a variable and was changed manually if need be.

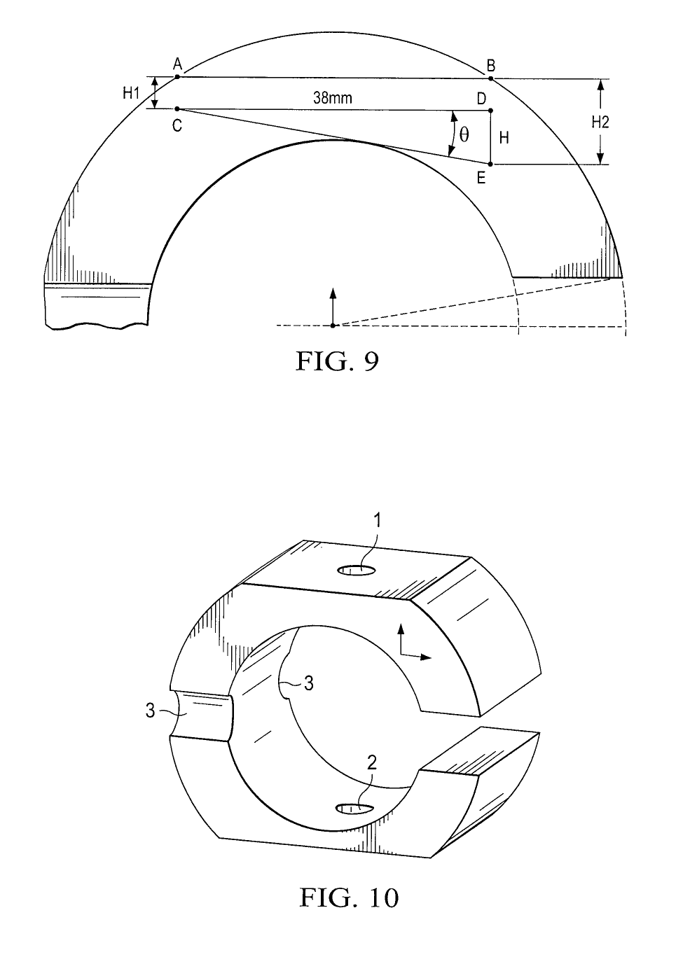

Simulations were conducted using the materials from Tables 1 and 2. The selected weight was for an approximately 180 lb. person. Each test produced stress and displacement diagrams used to determine where plastic deformation and ultimate failure might occur. Concentrated areas of stress where found on the posterior side of the ankle and on the lateral indentations. The top and front of the device were displaced most under compression. The angle of articulation (FIG. 9) was calculated. Results are shown in Table 3.

TABLE 3
Finite Element Analysis Results

| | Inner/outer | Maximum | Articulation | Maximum |
|---|---|---|---|---|
| Material (Yield | Diameter Sizes | displace- | Angle | Stress |
| Strength N/m2) | (inches) | ment(mm) | (estimated) | (N/m2) |
| ABS | 2.5/1 | 0.1 | small | — |
| 4.1e7 | 2.5/1 | 0.1 | small | — |
| | 3/1.5 | 2 | 1.6 | 3e7 |
| | 3.5/2.25 | 6.6 | 5.2 | 4e7 |
| White delrin | 2.5/1.5 | 4 | 3.2 | 4e7 |
| (POM) | | | | |
| 6.06e7 | 3/1.5 | 1.5 | 1.2 | 2.5e7 |
| | 3/2 | 7 | 5.5 | 5.3e7 |
| | 3.5/2.25 | 5 | 3.9 | 3.9e7 |
| Delrin AF | 2.5/1.5 | 4 | 3.2 | 5e7 |
| 1.1e8 | 3/1.5 | 1.4 | 1.1 | 2.7e7 |
| | 3/2 | 3.5 | 2.8 | 6.3e7 |
| | 3.5/2.25 | 4.7 | 3.7 | 4.5e7 |
| Delrin 2700 | 2.5/1.5 | 3.7 | 2.9 | 3.6e7 |
| (acetal | 3/1.5 | 1.3 | 1.0 | 2.2e7 |
| copolymer) | | | | |
| 6.3e7 | 3/2 | 6 | 4.7 | 5.6e7 |
| | 3.5/2.25 | 4.5 | 3.5 | 3.6e7 |
| High density PE | 2.5/1.5 | 10 | 7.8 | 4.4e7 |
| 2.3e7 | 3/1.5 | 3.7 | 2.9 | 2.3e7 |
| | 3/2 | 17 | 13.2 | 5.7e7 |
| | 3.5/2.25 | 13 | 10.2 | 4.1e7 |
| Low density PE | 2/1 | 35 | 25.9 | 3.3e7 |
| 1.45e7 | 2/0.75 | 16 | 12.4 | 2.1e7 |
| | 2/0.5 | 7 | 5.5 | 1.5e7 |
| | 3/1.5 | 27.0 | 20.5 | 2.7e7 |
| | 3/2 | large | | large |

Four material/dimension combinations were selected for further evaluation. The selection criteria were: a reasonable degree of articulation (approximately 3-10°), a maximum stress below the yield strength, the reported machinability of the plastic, and the cost of raw plastic rods.

Further testing examined the off-center distance of the center hole. Results are shown in Table 4. Moving the hole backward caused much greater stress on the ankle while moving it forward had the opposite effect. Moving the hole effectively adjusts the thickness of the load-bearing part of the device. Thus, the hole may be moved depending on the amputee's needs, or the posterior part of the device may be sanded or made a given thickness depending on these needs. The amputee's needs with respect to this feature may be determined, for example, by the amputee's weight.

TABLE 4
Offcenter Distance of Center Hole

| | | | | Artic- | |
|---|---|---|---|---|---|
| Material | Outer/inner | | Maximum | ulation | Max- |
| (Yield | Diameter | | Dis- | Angle | imum |
| Strength | Sizes | Offcenter | placement | (degrees, | Stress |
| N/m2) | (inches) | Distance | (mm) | estimated) | (N/m2) |
| White | 3/2 | 0″ | 6.8 | 5.4 | 5.3e7 |
| Delrin | | | | | |
| (POM) | | | | | |
| 6.06e7 | | −⅛″ | 15.4 | 12.5 | 1.005e8 |
| | | ⅛″ | 3.5 | 2.7 | 3.135e7 |
| Delrin | 3/2 | 0″ | 6.2 | 4.9 | 5.6e7 |
| 2700 | | | | | |
| (acetal | | −⅛″ | 14.1 | 11.4 | 1.064e8 |
| copolymer) | | | | | |
| 6.3e7 | | ⅛″ | 3.4 | 2.7 | 3.521e7 |
| | 3.5/2.25 | 0″ | 4.6 | 3.6 | 3.6e7 |
| | | −⅛″ | 8.8 | 7.0 | 6.61e7 |
| | | ⅛″ | 2.7 | 2.2 | 2.95e7 |
| High | 2.5/1.5 | 0″ | 10.0 | 8.0 | 4.4e7 |
| density PE | | | | | |
| 2.3e7 | | −⅛″ | 21.8 | 18.3 | 8.17e7 |
| | | ⅛″ | 5.9 | 4.7 | 2.56e7 |

### Example 2

### Physical Prototypes

After optimizing the performance of the virtual model by defining the dimensions and selecting the material the several physical prototypes were created. Material was acquired in rods from McMaster-Carr Supply Company. Four foot-long rods of the following materials were ordered: HDPE (diameter=2.5″), white Delrin (diameter=3″), and acetyl copolymer/Delrin 2700 (diameters=3″ and 3.5″). Once the plastic was received the device was first fabricated using the manual machines in a machine shop.

Rods were cut into separate pieces with thickness of roughly 1.75″. An automatic band saw was used in both machine shop and prosthetics facility to cut the pieces. This step may be skipped if the plastic is ordered cut to desired lengths.

The top and bottom faces of the device were sanded to have edge lengths of 2″. This provides a resulting piece with a good griping place for clamps in later steps. In the machine shop a milling machine with a drill bit was used for this step. It is important to note one must move the drill such that it cuts in the same direction as the spin direction first and then cuts inversely. This is to ensure an easier and cleaner cut. An electric sander may be used instead. Edges of the parts to be sanded down were labeled with a marker and the plastic piece was pressed against the sander until the marked edge was reached. The sanding process was faster than milling, but not as accurate. Using either method, the top and bottom part faces did not have to be perfectly parallel. Prosthesists can accommodate this variation during fitting.

The center hole was then drilled, which required a drill bit to take out a large amount of the material. The device was secured by clamping the top and bottom flat surfaces, and it was drilled through by a mill in the machine shop and a drill press. The diameter of the center hole was created by the diameter of the drill bit. Initially the dimensions determined virtually were followed, but some models did not seem practical after drilling. For example, after drilling a 1.5″ hole from a 2.5″ diameter rod with parts sanded off the top and bottom plates were less than ½″ thick. The thin walls raised concerns that they may induce material failing faster than predicted, so some modifications were made during the drilling process. Five models were made: HDPE with inner diameters of 1″ and 1.25″; while Delrin with 1.25″ and 1.5″; and Delrin 2700 with 1.25″. Delrin was a hard durable material, which created some difficulty for drilling. Each piece was completely clamped down for safety and consistency. It was also important to change the setting of drill press to low speed and high torque.

Two holes were then drilled at the top and bottom surface for the bolts. Just as drilling the center hole, these holes were drilled by a mill in the machine shop and a drill press. The diameter of the drill bit was 7/16″, which allowed insertion of the standard bolts used on a SACH foot. However, during the assembly process, it was discovered that the top hole had to be wider to enable fitting a bolt through from the center hole. Thus, the top hole was drilled using a step bit—a drill bit that increased in diameter up the bit. The top hole was thus more cone-shaped, starting from ½″ in diameter. Because prosthetists custom fit each ankle, the two holes do not have to be concentric.

The next step was to cut the gap. The location of the gap was not critical; it could be in the middle or farther up or down the anterior section of the ankle. The length of the gap was found to provide a maximum for how much the ankle could bend, in some cases. To provide a maximum allowance of 20 degrees, the length of the gap was determined using trigonometric properties and the known radius of the device. The length was 0.75″ for a 3″ diameter rod. The cut was made using a hacksaw or a band saw in the machine shop. The band saw provided more accuracy for determining the length and also cleanliness of cut; however, these were not important features for the ankle.

The posterior section of the device was then sanded/trimmed down. This section, across from the gap, was sanded down to allow more articulation. The amount to sand/trim down was arbitrary and was not always necessary, depending on the amputee. The back part was trimmed using a mill or the back part was simply sanded down. There is a relationship between patient weight and the thickness of the residual wall.

Lateral bites were then cut in the back wall. The bites were cut with a mill and sanded down.

### Example 3

### Prototype Evaluation

The finite element analysis results or Example 1 were validated with compression testing on one of the white Delrin prototypes. The compression machine, an ATF machine, collected data for load and displacement, which allowed for the creation of a load versus displacement graph (FIG. 18). At 200 pounds of force the ankle was displaced 0.055 inches (1.397 mm), which corresponds to a calculated 2.1 degree angle of deflection. This is comparable to the value of 1.5 mm (2.25 degrees) found with virtual application of 200 pounds via finite element analysis. Differences could be attributed to slight imperfections in the fabricated prototype.

For clinical testing a patient was fitted with a 3″ outer diameter 1.25″ inner diameter white Derlin prototype ankle. The patient reported good ankle function and significant dorsiflexion. The patent was able to both walk and jump using the prototype ankle. When the patient stood on one leg, placing all weight on the prosthetic ankle, a decrease in gap width of 0.021 inches was measured. Through geometric calculations, the angle of flexion was estimated to be approximately 0.8 degrees.

To prevent the possibility that the bolt attaching the ankle to the foot might strip out due to the softness of the plastic a tee nut which provided metal threading for the foot bolt to screw into was used. However, the curvature of the ankle's interior surface proved to be a problem when attempting to install the metal tee nut insert, which was meant for implantation into flat surfaces. Bending of the tee nut to conform to the curved surface compromised its shape and in turn its function.

In some examples the device was prone to rotation on top of the foot. To prevent this, double-sided sand paper and epoxy were used between the ankle and the foot, in addition to appropriate torque on the foot bolt. Before permanent placement however, it is important for ankle flexion to be aligned with the foot.

While embodiments of this disclosure have been depicted, described, and are defined by reference to specific example embodiments of the disclosure, such references do not imply a limitation on the disclosure, and no such limitation is to be inferred. The subject matter disclosed is capable of considerable modification, alteration, and equivalents in form and function, as will occur to those ordinarily skilled in the pertinent art and having the benefit of this disclosure. The depicted and described embodiments of this disclosure are examples only, and are not exhaustive of the scope of the disclosure.

1. A prosthetic articulating ankle joint comprising a horseshoe shaped component comprising:
an anterior gap to allow dorsiflexion;
a posterior portion;
at least one lateral indentation in the posterior portion to allow eversion, inversion or torsion;
at least one orifice in a top to allow attachment to a pylon; and
at least one orifice in a bottom to allow attachment to a prosthetic foot.

2. The prosthetic articulating ankle joint of claim 1, wherein the horseshoe shaped component is a C-shaped component.

3. The prosthetic articulating ankle joint of claim 2, wherein the C-shaped component comprises:
a top anterior projection;
a bottom anterior projection; and
a gap between the top anterior projection and the bottom anterior projection.

4. The prosthetic articulating ankle joint of claim 3, wherein the top and bottom anterior projections of the C-shaped component prevent dorsiflexation.

5. The prosthetic articulating ankle joint of claim 3, wherein the top and bottom anterior projections of the C-shaped component reduce dorsiflexation.

6. The prosthetic articulating ankle joint of claim 3, wherein the gap between the top and bottom anterior projection closes upon itself upon exposure of the prosthetic articulating ankle joint to a large force.

7. The prosthetic articulating ankle joint of claim 1, comprising an open interior wherein the anterior gap allows compression during the dorsiflexation.

8. The prosthetic articulating ankle joint of claim 1, wherein the orifice in the bottom to fit a prosthetic foot has a universal-fit design.

9. The prosthetic articulating ankle joint of claim 1, wherein the prosthetic foot is a non-articulating foot.

10. The prosthetic articulating ankle joint of claim 9, wherein the non-articulating foot is a solid ankle cushion heel foot (SACH foot).

11. The prosthetic articulating ankle joint of claim 1, wherein the prosthetic foot is an articulating foot.

12. The prosthetic articulating ankle joint of claim 11, wherein the articulating prosthetic foot is selected from a group consisting of TruStep, Venture, Tribut or a TruPer.

13. The prosthetic articulating ankle joint of claim 1, wherein the prosthetic foot is an energy storing foot.

14. The prosthetic articulating ankle joint of claim 13, wherein the energy storing foot is a Flex-foot.

15. The prosthetic articulating ankle joint of claim 1, comprised of one or more materials selected from the group consisting of high density polyethylene (HDPE), acrylonitrile butadiene styrene (ABS), Nylon, white Delrin, polypropylene, or polyethylene.

16. The prosthetic articulating ankle joint of claim 1, wherein the orifice in the top to fit a pylon has a universal-fit design.

17. A prosthetic limb comprising:
a prosthetic foot;
a pylon; and
a horseshoe shaped prosthetic articulating ankle joint comprising:
an anterior gap to allow dorsiflexion;
a posterior portion;
at least one lateral indentation in the posterior portion to allow eversion or inversion;
at least one orifice in a top to allow attachment to the pylon; and
at least one orifice in a bottom to allow attachment to the prosthetic foot.

18. The prosthetic limb of claim 17, wherein the horseshoe shaped prosthetic articulating ankle joint is C-shaped.

19. A prosthetic articulating ankle joint comprising a horseshoe shaped component comprising:
a means to provide dorsiflexion;
a means to provide eversion or inversion;
a means for attachment to a pylon; and
a means for attachment to a foot.

20. The prosthetic articulating ankle joint of claim 19, wherein the horseshoe shaped component is C-shaped.
